(19) United States
(12) Patent Application Publication
(10) Pub. No.: US 2026/0130948 A1
(43) Pub. Date: May 14, 2026

(54) RADIATION DAMAGE PROTECTION AGENT

(71) Applicant: JICHI MEDICAL UNIVERSITY, Tokyo (JP)

(72) Inventors: Kotaro YOSHIMURA, Tochigi (JP); Yoshihiro SOWA, Tochigi (JP)

(73) Assignee: JICHI MEDICAL UNIVERSITY, Tokyo (JP)

(21) Appl. No.: 18/878,755

(22) PCT Filed: Jul. 4, 2023
(86) PCT No.: PCT/JP2023/024730

§ 371 Date: Dec. 29, 2025

(30) Foreign Application Priority Data
Jul. 4, 2022 (JP) 2022-107975

(51) Int. Cl. A61K 35/35; A61P 17/16

(52) U.S. Cl. CPC A61K 35/35; A61P 17/16

(57) ABSTRACT

The object of the present invention is to provide a novel radiation damage protective agent. The present invention provides a radiation damage protective agent which comprises adipose tissue or adipose tissue-derived product (for example, Stromal vascular fraction (SVF), micronized cellular adipose matrix (MCAM), or Adipose-derived stem/stromal cells (ASC)).

### TECHNICAL FIELD

The present invention relates to a radiation damage protective agent which comprises adipose tissue or adipose tissue-derived products.

### BACKGROUND ART

Radiation therapy is one of the primary treatments for preventing cancer proliferation and recurrence. Although not common with current clinical radiation therapy dosing regimens, the incidence of skin malignancies increases, depending on the radiation dose (Non-Patent Documents 1 to 3). In contrast, definitive effects occur in surrounding healthy tissues. The skin is significantly affected by radiation exposure, exhibiting changes such as fibrosis, atrophy, microvascular occlusion (ischemia), and skin thickening (Non-Patent Documents 2-6). Such radiation-induced damage leads to various clinical manifestations in the skin and subcutaneous tissue, including radiation dermatitis, scar contracture, lymphedema, and difficult wound healing. Furthermore, skin elasticity and extensibility are impaired, skin appendages and hair follicles are lost, and joint movement is restricted (Non-Patent Document 5).

Fractionated radiation therapy has been developed to maximize therapeutic efficacy while reducing deterministic effects on healthy tissue; however, long-term tissue damage still occurs (Non-Patent Document 7). Impairment of tissue renewal/remodeling and wound healing after radiation therapy can progress to tissue dysfunction, potentially resulting in severe symptoms such as refractory skin ulcers or osteomyelitis years after radiation exposure (Non-Patent Documents 1, 2, 6, and 7).

On the other hand, adipose tissue and its mesenchymal stem cells (adipose-derived stem cells, ASCs) have been demonstrated to possess angiogenic and regenerative capabilities (Non-Patent Documents 8-13). The inventors previously showed that ASCs and adipose-derived products containing ASCs can partially restore wound healing capacity in radiation-damaged rats (Non-Patent Document 14).

### PRIOR ART DOCUMENTS

### Non-Patent Documents

Non-Patent Document 1: Martin M T, Vulin A, Hendry J H. Human epidermal stem cells: role in adverse skin reactions and carcinogenesis from radiation. Mutat Res. 2016; 770:349-368.
Non-Patent Document 2: Spalek M. Chronic radiation-induced dermatitis: challenges and solutions. Clin Cosmet Investig Dermatol. 2016; 9:473-482.
Non-Patent Document 3: Bray F N, Simmons B J, Wolfson A H, Noun K. Acute and chronic cutaneous reactions to ionizing radiation therapy. Dermatol Ther (Heidelb). 2016; 6:185-206.
Non-Patent Document 4: Kim J H, Kolozsvary A J, Jenrow K A, et al. Mechanisms of radiation-induced skin injury and implications for future clinical trials. Int J Radiat Biol. 2013; 89:311-318.
Non-Patent Document 5: Zawaski J A, Yates C R, Miller D D, et al. Radiation combined injury models to study the effects of interventions and wound biomechanics. Radiat Res. 2014; 182:640-652.
Non-Patent Document 6: Soriano J L, Calpena A C, Souto E B, Clares B. Therapy for prevention and treatment of skin ionizing radiation damage: a review. Int J Radiat Biol. 2019; 95:537-553.
Non-Patent Document 7: Jagetia G C, Rajanikant G K. Acceleration of wound repair by curcumin in the excision wound of mice exposed to different doses of fractionated y radiation. Int Wound J. 2012; 9:76-92.
Non-Patent Document 8: Maria O M, Shalaby M, Syme A, Eliopoulos N, Muanza T. Adipose mesenchymal stromal cells minimize and repair radiation-induced oral mucositis. Cytotherapy. 2016; 18:1129-45.
Non-Patent Document 9: Wu S H, Shirado T, Mashiko T, et al. Therapeutic Effects of Human Adipose-Derived Products on Impaired Wound Healing in Irradiated Tissue. Plast Reconstr Surg. 2018; 142:383-391.
Non-Patent Document 10: Nauta A, Seidel C, Deveza L, et al. Adipose-derived stromal cells overexpressing vascular endothelial growth factor accelerate mouse excisional wound healing. Mol Ther. 2013; 21:445-455.
Non-Patent Document 11: Izadpanah R, Trygg C, Patel B, et al. Biologic properties of mesenchymal stem cells derived from bone marrow and adipose tissue. J Cell Biochem. 2006; 99:1285-1297.
Non-Patent Document 12: Zhao L, Johnson T, Liu D. Therapeutic angiogenesis of adipose-derived stem cells for ischemic diseases. Stem Cell Res Ther. 2017; 8:125.
Non-Patent Document 13: Hassan W U, Greiser U, Wang W. Role of adipose-derived stem cells in wound healing. Wound Repair Regen. 2014; 22:313-325.
Non-Patent Document 14: Feng J, Doi K, Kuno S, et al. Micronized cellular adipose matrix as a therapeutic injectable for diabetic ulcer. Regen Med. 2015; 10:699-708.
Non-Patent Document 15: Yoshimura K, Shigeura T, Matsumoto D, et al. Characterization of freshly isolated and cultured cells derived from the fatty and fluid portions of liposuction aspirates. J Cell Physiol. 2006; 208:64-76.
Non-Patent Document 16: H. Eray Copcu, MD; and Sule Oztan, MD. New Mechanical Fat Separation Technique: Adjustable Regenerative Adipose-tissue Transfer (ARAT) and Mechanical Stromal Cell Transfer (MEST) Aesthetic Surgery Journal Open Forum 2020, 1-15.
Non-Patent Document 17: Moore G H, Schiller J E, Moore G K. Radiation-induced histopathologic changes of the breast: the effects of time. Am J Surg Pathol. 2004; 28:47-53.
Non-Patent Document 18: Bolderston A, Lloyd N S, Wong R K, Holden L, RobbBlenderman L. Supportive Care Guidelines Group of Cancer Care Ontario Program in Evidence-Based Care. The prevention and management of acute skin reactions related to radiation therapy: A systematic review and practice guideline. Support Care Cancer 2006; 14:802-817.
Non-Patent Document 19: Thanik V D, Chang C C, Zoumalan R A, et al., A novel mouse model s of cutaneous radiation injury. Plast Reconstr Surg. 2011; 127:560-568.
Non-Patent Document 20: Sitton E. Early and late radiation-induced skin alterations. Part I: Mechanisms of skin changes. Oncol Nurs Forum. 1992; 19:801-807.
Non-Patent Document 21: López E, Guerrero R, Núñez M I, et al. Early and late skin reactions to radiotherapy for breast cancer and their correlation with radiation-induced DNA damage in lymphocytes. Breast Cancer Res. 2005; 7:690-698.
Non-Patent Document 22: Zhou Y, Zhang Y. Single versus 2-stage reconstruction for chronic postradiation chest wall ulcer: A 10-year retrospective study of chronic radiation-induced ulcers. Medicine (Baltimore). 2019; 98:e14567.
Non-Patent Document 23: Azuma R, Kajita M, Kubo S, Kiyosawa T. Radiation-induced thoracic necrosis with a pulmonary cutaneous fistula repaired using a free omental flap: a case report. BMC Surg. 2019; 19:14.
Non-Patent Document 24: Mohd Hilmi A B, Halim A S, Jaafar H, Asiah A B, Hassan A. Chitosan dermal substitute and chitosan skin substitute contribute to accelerated full-thickness wound healing in irradiated rats. Biomed Res Int. 2013; 2013:795458.
Non-Patent Document 25: Rigotti G, Marchi A, Gali e M, et al. Clinical treatment of radiotherapy tissue damage by lipoaspirate transplant: a healing process mediated by adipose derived adult stem cells. Plast Reconstr Surg. 2007; 119:1409-1424.
Non-Patent Document 26: Copcu H E, Oztan S. Not Stromal Vascular Fraction (SVF) or Nanofat, but Total Stromal-Cells (TOST): A New Definition. Systemic Review of Mechanical Stromal-Cell Extraction Techniques. Tissue Eng Regen Med. 2021; 18:25-36.
Non-Patent Document 27: Yao Y, Dong Z, Liao Y, et al. Adipose Extracellular Matrix/Stromal Vascular Fraction Gel: A Novel Adipose Tissue-Derived Injectable for Stem Cell Therapy. Plast Reconstr Surg. 2017; 139:867-879.

### SUMMARY OF THE INVENTION

### Problem to be Solved by the Invention

Radiation therapy is currently a central treatment for malignant tumors, but it has the potential to induce definitive adverse effects on surrounding healthy tissue, such as atrophy, fibrosis, ischemia, and impaired wound healing. The present invention addresses the challenge of providing a novel radiation damage protective agent.

### Means for Solving the Problem

The present inventors have investigated whether prophylactic administration of a composition containing adipose tissue-derived stem cells immediately after radiation therapy could prevent the onset of long-term functional impairment in irradiated tissues. Specifically, nude mice received a total irradiation dose of 40 Gy (10 Gy, four times weekly) to the dorsal skin. Vehicle, adipose tissue, stromal vascular fraction (SVF), or minced adipose tissue matrix (MCA) were then subcutaneously injected into the irradiated area. Six months after these preventive treatments, skin punch wounds were created to evaluate histological changes and wound healing. The results showed that histological evaluation demonstrated increased skin thickening, atrophy, and collagen deposition in the subcutaneous fat layer six months after radiation therapy in all preventive treatment groups, with significantly delayed wound healing. Compared to the vehicle, prophylactic treatment with adipose tissue-derived products containing human adipose stem cells significantly prevented radiation-induced histological changes and accelerated wound healing. The present invention was completed based on the above findings.

Accordingly, the present invention provides the following invention.
<1> A radiation damage protective agent which comprises adipose tissue or an adipose tissue-derived product.
<2> The radiation damage protective agent of <1>, wherein the radiation damage is chronic radiation damage.
<3> The radiation damage protective agent of <1> or <2>, wherein the radiation damage is a decrease in wound healing ability.
<4> The radiation damage protective agent of any one of <1> to <3>, which has an action to prevent subcutaneous tissue atrophy.
<5> The radiation damage protective agent of any one of <1> to <4>, wherein the fat tissue-derived product is stromal vascular fraction (SVF), micronized cellular adipose matrix (MCAM), or adipose-derived stem/stromal cells (ASC), or a culture supernatant of SVF, MCAM or ASC, or an extract of SVF, MCAM or ASC.
<6> The radiation damage protective agent of any one of <1> to <5>, which is administered to a subject immediately after radiation exposure.
<7> The radiation damage protective agent of any one of <1> to <6>, wherein the adipose tissue or the adipose tissue-derived product is autologous or allogeneic.
<A> A method for protecting against radiation damage, which comprises administering adipose tissue or adipose tissue-derived product to a subject.
<B> Adipose tissue or adipose tissue-derived products for use in treatment of protecting against radiation damage.
<C> Use of adipose tissue or adipose tissue-derived product for production of a radiation damage protective agent.

### Effect of the Invention

According to the present invention, a novel radiation damage protective agent is provided.

### BRIEF DESCRIPTION OF THE DRAWINGS

FIG. 1 shows the timeline of the experimental plan. After radiation exposure (4 fractions of 10 Gy), three types of adipose tissue-derived products were subcutaneously injected into the irradiated area. Six months later, circular wounds were created and observed for 15 days.

FIG. 2 shows the irradiation settings. The body was protected from irradiation with lead covers, except for the irradiation field and the ears.

FIG. 3 shows photographs immediately after radiation therapy. As a result of acute radiation injury, hair loss was induced in the irradiation field.

FIG. 4 shows the prophylactic injection of adipose tissue-derived product. As prophylactic treatment to prevent chronic radiation damage, three types of human adipose tissue-derived products were injected using an 18G needle.

FIG. 5 shows the preventive effect of human adipose tissue-derived products on wound healing after radiation therapy. Representative images of wound healing in each group are shown. Six months after radiation therapy and prophylactic administration of human adipose tissue-derived products, wounds (6 mm diameter) were created via punch biopsy. Wound size was measured digitally using ImageJ software at days 0, 3, 6, 9, 12, and 15.

FIG. 6 shows the quantitative evaluation of wound healing. In the solvent group, no reduction in wound size was observed during the initial 6 days. However, in the groups administered the three types of human adipose tissue-derived products, wound healing was accelerated, and by day 15, the wounds were almost completely healed, comparable to the non-radiation-treated control group. Data are expressed as median with interquartile range (IQR). (*) P<0.05 and (**) P<0.01 vs. R+solvent group; +P<0.05 vs. SVF group; n=6 per group.

FIG. 7 shows histological evaluation of irradiated tissue at 6 months with and without preventive treatment. Representative micro-sections stained with hematoxylin and eosin are shown. In the vehicle-treated group, the dermis was thickened, and the fat layer was atrophied as a result of radiation therapy. Scale bar indicates 200 μm.

FIG. 8 shows quantitative data on dermal and adipose layer thickness. The groups administered adipose tissue-derived products did not exhibit the dermal thickening seen in the solvent-treated group, demonstrating a preventive effect. Furthermore, the severe adipose layer atrophy observed in the vehicle-treated group was partially prevented by the three types of adipose-derived products used for prophylactic treatment. Data are shown as the median IQR. Horizontal lines on the bars and P-values indicate statistically significant differences (P<0.05) between groups.

FIG. 9 shows the histological evaluation of subcutaneous fibrosis in irradiated tissue at 6 months, with and without preventive treatment. Representative micro-sections stained with Masson's trichrome are shown. Collagen deposits were stained blue with Masson's trichrome. Scale bar indicates 200 μm.

FIG. 10 shows quantitative data on collagen deposition in the adipose layer. The percentage of collagen deposits within the adipose layer was calculated. The most severe fibrosis was observed in the vehicle-treated irradiation group, but it was significantly prevented in groups pretreated with fat, SVF, or MCAM. Among the three adipose tissue-derived product treatment groups, the Fat group showed a higher percentage of fibrosis than the SVF group. Data are expressed as median and interquartile range (IQR). P values are shown when a significant difference (P<0.05) was observed between groups.

FIG. 11 shows a clinical scenario for prophylactic treatment using human adipose tissue-derived products to prevent radiation-induced skin injury. This example validated a novel therapeutic stem cell therapy based on the concept of preventing radiation-induced stem cell depletion. Prophylactic injection of aspirated adipose tissue-derived or processed ASC concentrates at an early stage after radiation therapy can minimize radiation injury. Preventive treatment immediately following radiation therapy can be used to avoid fibrosis, ischemia, and impaired healing. It was demonstrated that this represents a more appropriate approach than subsequent treatment if long-term adverse effects induced by radiation therapy are observed.

FIG. 12 shows adipose tissue-derived products It shows the appearance and microscopic images of aspirated adipose tissue (left) and minced adipose tissue matrix (MCA; right). The scale bar indicates 100 μm.

FIG. 13 shows irradiation of mice. The left image depicts targeting the irradiated mouse area using an X-ray generator and lead plates. The right image shows prevention of wound contracture using a donut-shaped silicone splint (9 mm diameter).

FIG. 14 shows histological and immunohistochemical findings 6 months after radiotherapy with and without prophylactic treatment. (A) shows immunostaining for perilipin alone (left, green, surviving adipocytes) or nuclear staining (right, blue). Scale bar indicates 100 μm. (B) shows quantitative measurement of surviving adipocytes in the adipose layer. Radiation therapy induced fat atrophy, which was significantly prevented by prophylactic treatment with SVF or MCAM. Data are shown as the median IQR

### EMBODIMENTS FOR CARRYING OUT THE INVENTION

The following describes specific embodiments of the present invention. The description below are provided to facilitate understanding of the invention. The scope of the invention is not limited to the embodiments described below. Other embodiments, in which a person skilled in the art appropriately substitutes the configurations of the embodiments described below, are also included within the scope of the invention.

According to the present invention, the possibility of preventive treatment after radiation therapy is demonstrated, enabling the prevention of worsening chronic radiation damage. The radiation damage protective agent of the present invention is useful in radiation therapy. Preventive treatment using the radiation damage protective agent of the present invention has the potential to improve wound healing in irradiated tissue and the clinical outcomes of reconstructive surgery required after cancer radiation therapy.

The radiation damage protective agent of the present invention comprises adipose tissue or adipose tissue-derived product.

The adipose tissue-derived product is not particularly limited, but include, for example, Stromal vascular fraction (SVF), micronized cellular adipose matrix (MCAM), or adipose-derived stem/stromal cells (ASC). Alternatively, culture supernatant of SVF, MCAM, or ASC, or extract of SVF, MCAM or ASC may be used. The adipose tissue or adipose tissue-derived product may be autologous or allogeneic.

Adipose tissue is one type of connective tissue that constitutes the body of living organisms and is primarily located subcutaneously. Adipose tissue mainly contains mature adipocytes and possesses functions such as storing energy, protecting the body from external physical impacts and temperature changes, and secreting hormones, cytokines and other substances. In this specification, adipose tissue may be referred to as fat.

The Stromal vascular fraction (SVF) refers to a population of cells isolated by fragmenting adipose tissue, followed by enzymatic treatment and filtration. This fraction contains adipose stem cells, vascular endothelial (precursor) cells, leukocytes, fibroblasts, and others. Adipocytes and matrix (extracellular matrix) are removed during the manufacturing process.

Micronized cellular adipose matrix (MCAM) is an extracellular matrix fraction obtained by micronizing adipose tissue and further subjecting it to mechanical disruption. It contains adipose stem cells, vascular endothelial (precursor) cells, fibroblasts, and other cells within the fibrous tissue. Adipocytes are removed during the manufacturing process.

Adipose-derived stem/stromal cells (ASC) are somatic stem cells derived from adipose tissue, referring to cells satisfying the following definitions (1) to (4).

### Definition of Adipose-Derived Stem Cells

(1) Derived from adipose tissue.
(2) They exhibit adhesion to plastic under standard culture conditions.
(3) They are positive for CD90, CD73, and CD105 by flow cytometry.
(4) They are negative for CD31 and CD45 by flow cytometry.

Adipose-derived stem cells may possess differentiation potential into adipocytes, osteoblasts, chondrocytes, myofibroblasts, osteoblasts, muscle cells, or nerve cells.

CD90 refers to differentiation cluster 90 which is one type of surface antigens, and is a protein also known as Th-1.

CD73 refers to differentiation cluster 73 which is one type of surface antigens, and is a protein also known as 5-Nucleotidase or Ecto-5′-nucleotidase.

CD105 refers to differentiation cluster 105 which is one type of surface antigens, and is a protein also known as Endoglin.

CD31 refers to differentiation cluster 31 which is one type of surface antigens, and is a protein also known as PECAM-1 (Platelet Endothelial Adhesion Molecule-1).

CD45 refers to differentiation cluster 45 which is one type of surface antigen, and is a protein also known as PTPRC (Protein Tyrosine Phosphatase, receptor type, C), or LCA (Leukocyte common antigen).

The adipose-derived stem cells are preferably passaged adipose-derived stem cells. The adipose-derived stem cells may be autologous, allogeneic, or xenogeneic cells, but are preferably autologous cells. The adipose-derived stem cells are preferably non-genetically modified adipose-derived stem cells. The adipose-derived stem cells may be commercially available cells, cells obtained through distribution, or newly prepared cells. The adipose-derived stem cells may be isolated adipose-derived stem cells. The adipose-derived stem cells may be selected adipose-derived stem cells.

The adipose-derived stem cells may preferably be adipose-derived stem cells which have undergone at least one adhesion culture. The lower limit for the number of adhesion cultures of the adipose-derived stem cells may be one or more times, two or more times, three or more times, four or more times, five or more times, or six or more times. Furthermore, the upper limit of the number of adhesion cultures of the adipose-derived stem cells is not particularly limited, but may be, for example, 25 times or less, 20 times or less, 15 times or less, or 10 times or less.

The species from which the adipose tissue is derived is typically human, but may also be other animals. Examples of other animals include dogs, cats, cattle, horses, pigs, goats, sheep, monkeys (crab-eating macaques, rhesus macaques, common marmosets, Japanese macaques), ferrets, rabbits, rodents (mice, rats, gerbils, guinea pigs, hamsters), birds such as chickens and quails, but are not limited to these.

The protective effect against radiation damage of the present invention is thought to be due to the efficacy of secretions from ASCs. Therefore, when using the culture supernatant of SVF, MCAM or ASC or the extract of SVF, MCAM or ASC, it is expected that a protective effect similar to that achieved when using SVF, MCAM, or ASCs can be attained.

When using SVF or MCAM, ASC may be isolated from the SVF or MCAM and may be cultured. Then, the culture supernatant may be obtained. The culture supernatant of SVF, MCAM or ASC can be obtained by culturing them in a culture medium.

The culture medium is not particularly limited as long as it is a medium capable of culturing ASCs, and examples include EGM-2 (Lonza), αMEM, Dulbecco's Modified Eagle Medium (DMEM), Dulbecco's Modified Eagle Medium/Ham's F-12 Mixed Medium (DMEM/F12), RPMI 1640, etc. For these culture media, various additives applicable to conventional cell culture, such as serum, various vitamins, various antibiotics, various hormones, and various growth factors, may typically be added. Particularly preferred media include DMEM/F12, or EGM-2, EGM-2MV (both Lonza), etc.

Culture is preferably performed in culture vessels such as flasks at 5% CO₂ and 37° C. Medium changes may be performed, for example, every two days. Cultures can be maintained at oxygen concentrations of 1-21%. Cultures are particularly preferably maintained at oxygen concentrations of 2-6%. The culture period is not specifically limited, but cultures may be maintained, for example, for 1 to 14 days. After culturing for 1 to 6 days, the cells may be passaged and cultured again for, for example, 3 to 6 days. The number of passages and the number of culture cycles are not particularly limited.

In the culture supernatant acquisition step, the culture supernatant can be acquired from a culture of cells in the proliferative phase or confluent phase. The culture supernatant can be prepared by methods known in the art. For example, the culture supernatant can be obtained by centrifuging the obtained culture medium and passing it through a filter (or strainer) with an appropriate pore size. Here, centrifugation can be performed, for example, at 300 to 1200×g for 5 to 20 minutes.

The extract of SVF, MCAM or ASC can also be obtained by conventional extraction methods.

According to the present invention, a radiation damage protective agent which comprises adipose tissue or adipose tissue-derived products is provided. The radiation damage protective agent of the present invention may be provided as a pharmaceutical composition for radiation damage protection, which comprises adipose tissue or adipose tissue-derived product and a pharmaceutically acceptable carrier.

According to the present invention, adipose tissue or adipose tissue-derived product are provided for use in treatment of protecting against radiation damage.

According to the present invention, the use of adipose tissue or adipose tissue-derived product for production of a radiation damage protective agents is provided.

According to the present invention, a method for protecting against radiation damage is provided, which comprises administering a protectively effective amount of adipose tissue or adipose tissue-derived product to a patient or subject.

In the present invention, radiation includes alpha rays, beta rays, gamma rays emitted from radioactive substances, as well as artificially produced X-rays, proton beams, carbon beams, neutron beams, electron beams, etc. Causes of radiation exposure include, but are not limited to, systemic radiation exposure due to nuclear accidents or nuclear explosions, localized radiation exposure due to radiation irradiation for medical purposes such as cancer treatment or radiation exposure accidents, etc.

The radiation damage referred to in the present invention is preferably chronic radiation damage. An example of radiation damage includes impaired wound healing ability, but is not specifically limited thereto.

The radiation damage protective agent of the present invention preferably has an action that prevents subcutaneous tissue atrophy.

The subject (patient or subject) to whom the radiation damage protective agent of the present invention is administered is typically a human, but may also be an animal other than a human. Examples of animals other than humans include, for example, dogs, cats, cattle, horses, pigs, goats, sheep, monkeys (crab-eating macaques, rhesus macaques, common marmosets, Japanese macaques), ferrets, rabbits, rodents (mice, rats, gerbils, guinea pigs, hamsters), birds such as chickens and quails, but are not limited to these.

Pharmaceutically acceptable media refer to liquids that can be administered to patients or subjects. Pharmaceutically acceptable media are not particularly limited as long as they are liquids that can be administered to patients or subjects. Examples of pharmaceutically acceptable media include water for injection, physiological saline solution, culture media, 5% glucose solution, hyaluronic acid solution, Ringer's solution, Lactated Ringer's solution, Acetate Ringer's solution, Bicarbonate Ringer's solution, Bikanate® infusion, amino acid solution, initial solution (Solution No. 1), dehydration replacement solution (Solution No. 2), maintenance infusion (Solution No. 3), postoperative recovery solution (Solution No. 4), Plasma-Lyte® A, etc., but are not limited to these.

The radiation damage protective agent of the present invention may comprise any additive which can be administered to a patient or subject, and may comprise any additive which can adjust the storage stability, isotonicity, absorbency, and/or viscosity, etc., of the radiation damage protective agent. Examples of additives include, but are not limited to, emulsifiers, dispersants, buffers, preservatives, humectants, antioxidants, chelating agents, thickeners, gelling agents, and pH adjusters. Examples of thickeners include, but are not limited to, HES, dextran, methylcellulose, xanthan gum, carboxymethylcellulose, hydroxypropyl cellulose, and the like, but are not limited to these. The concentration of additives may be set arbitrarily as long as it is safe when administered to a patient or subject.

The radiation damage protective agent of the present invention may comprise any component which can be administered to a patient or subject. Examples of such components include salts, polysaccharides (e.g., hydroxyethyl starch (HES), dextran, etc.), proteins (e.g., albumin, etc.), dimethyl sulfoxide (DMSO), amino acids, culture medium components, etc., but are not limited to these.

The pH of the radiation damage protective agent of the present invention may be near neutral, for example, pH 5.5 or higher, pH 6.0 or higher, pH 6.5 or higher, or pH 7.0 or higher. It may also be pH 10.5 or lower, pH 9.5 or lower, pH 8.5 or lower, or pH 8.0 or lower, but is not limited to these.

The radiation damage protective agent of the present invention is preferably a liquid preparation, and more preferably an injectable liquid preparation. As injectable liquid preparations, liquid formulations suitable for injection are known, for example, in International Publication WO 2011/043136 and Japanese Patent Application Publication No. 2013-256510. The pharmaceutical composition of the present invention may also be an injectable liquid preparation described in the above Documents.

Furthermore, the liquid agent may be a cell suspension or a liquid preparation in which cells are dispersed within the liquid agent. Additionally, the morphology of the cells contained in the liquid agent may be, for example, single cells or cell aggregates.

The administration methods for the radiation damage protective agent of the present invention are not particularly limited, but include, for example, subcutaneous injection, intradermal injection, intramuscular injection, intralymphatic injection, intravenous injection, intra-arterial injection, intravenous drip injection, intraperitoneal injection, intrathoracic injection, direct injection into a specific site, and topical application to a specific site. According to one aspect of the present invention, the injectable solution can be filled into a syringe and administered via an injection needle or catheter. Regarding methods for administering pharmaceutical compositions, see, for example, Japanese Patent Application Publication No. 2015-61520, Onken J E, et al. American College of Gastroenterology Conference 2006 Las Vegas, NV, Abstract 121., Garcia-Olmo D, et al. Dis Colon Rectum 2005; 48:1416-23. Intravenous injection, intravenous drip, and direct local injection are known methods described in the above documents. The pharmaceutical composition of the present invention can also be administered by the various methods described in the above documents.

The dosage of the radiation damage protective agent of the present invention is any amount sufficient to protect against radiation damage when administered to a patient or subject. The specific dosage can be appropriately determined based on the dosage form, administration method, intended use, and the patient's or subject's age, weight, symptoms, etc.

The frequency of administration of the radiation damage protective agent of the present invention is the frequency at which an effect of protecting against radiation damage can be obtained when administered to a patient or subject. The specific administration frequency may be appropriately determined based on the dosage form, administration method, intended use, and the patient's or subject's age, weight, symptoms, etc. Examples include once every 4 weeks, once every 3 weeks, once every 2 weeks, once per week, twice per week, three times per week, four times per week, five times per week, six times per week, or seven times per week. Preferably, the radiation damage protective agent of the present invention can be administered to the subject immediately after exposure.

The radiation damage protective agent of the present invention is preferably administered immediately before or after radiation exposure. Specifically, administration may begin within 60 minutes before or after radiation exposure, preferably within 30 minutes, more preferably within 15 minutes, further preferably within 10 minutes, and particularly preferably within 5 minutes. Additionally, further administration may be performed one day or later after radiation exposure. Specifically, one or more additional doses (e.g., one dose, preferably two doses, more preferably three doses) may be administered one day or later after radiation exposure and within 10 days (more preferably within 8 days, particularly preferably within 7 days).

The radiation damage protective agent of the present invention can be stored in a frozen state until immediately before use. When administering the radiation damage protective agent of the present invention to a patient or subject, it can be rapidly thawed at 37° C. for use. Furthermore, the radiation damage protective agent of the present invention can also be used immediately after manufacture without frozen storage.

The present invention will be described in detail below with reference to the following examples, but the invention is not limited to these examples.

### EXAMPLES

### (1) Materials and Methods

### <Isolation and Preparation of Cells>

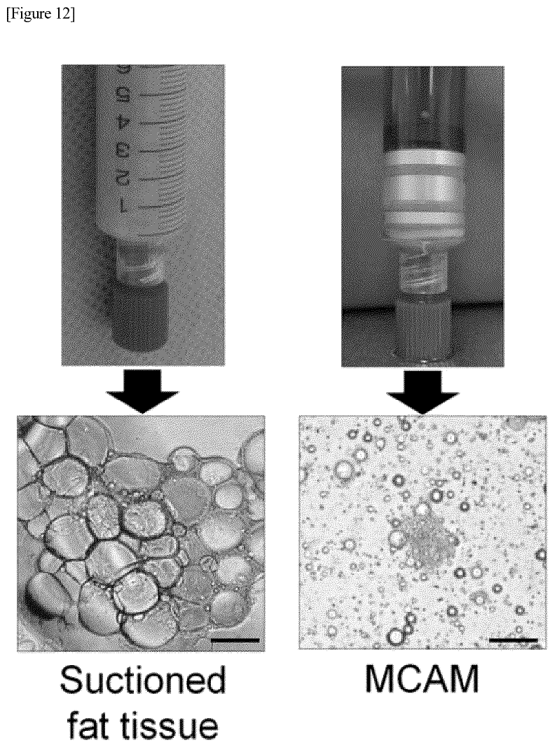

Excess adipose tissue following breast reconstruction was harvested from patients. Each donor provided prior informed consent according to a protocol approved by the Institutional Review Board (ERB-C-487-1). Human SVF cells were isolated from aspirated fat as previously reported (Non-Patent Document 15). Briefly, lipoaspirate was washed with phosphate-buffered saline (PBS), digested in PBS containing 0.075% collagenase, and shaken at 37° C. for 30 minutes. Mature adipocytes and connective tissue were separated from the cell pellet by centrifugation at 800×g for 10 minutes. The pellet was rinsed, filtered through a 100 μm mesh, resuspended, and dispersed for culture at 37° C. in 5% carbon dioxide (1.0×10⁶ nucleated cells per 100 mm dish). The minced adipose tissue matrix (MCAM) was prepared using a sharp blade system (Adinizer®, BSL, Gimhae, Korea) (Non-Patent Document 16). The centrifuged fat was manually transferred ten times between two syringes using the adipose tissue micro-mincer to gently separate mature adipocytes from the interstitial tissue. Two types of adipose tissue mincing devices, AN-1200P and AN-410, were used consecutively in this process. Furthermore, MCAM was purified using centrifugation at 800×g for 3 minutes (FIG. 12) (Non-Patent Documents 9, 14, 16).

<Animal Model of Radiation Exposure and Prophylactic Injection of Processed Products Prepared from Adipose Tissue>

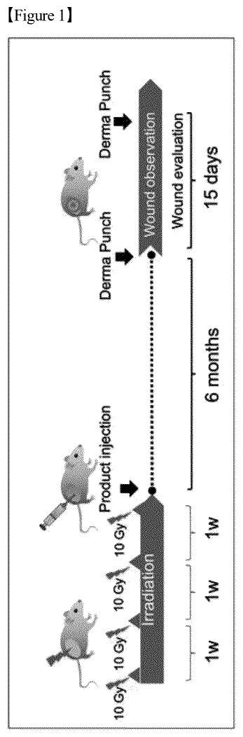

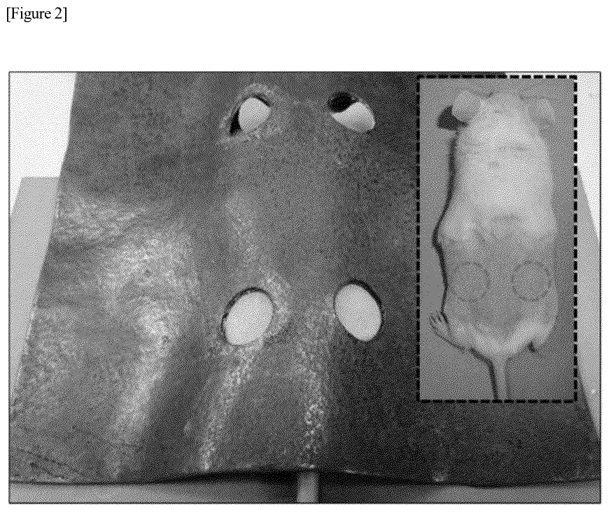

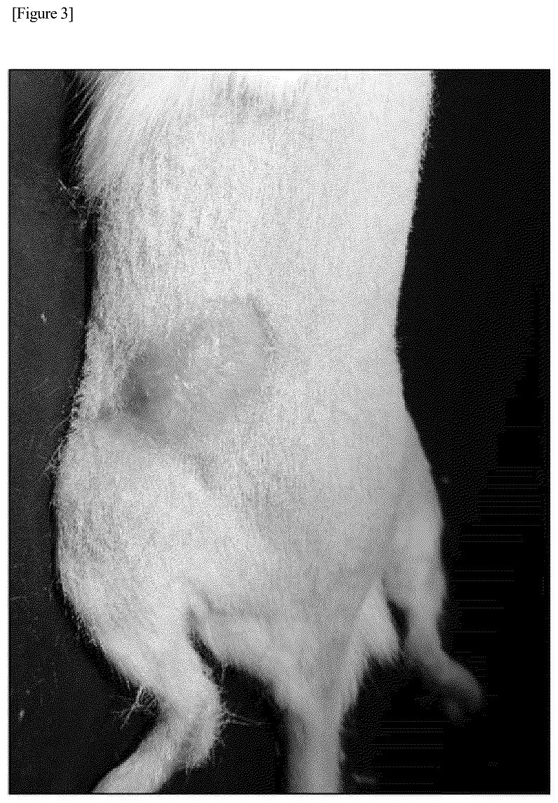

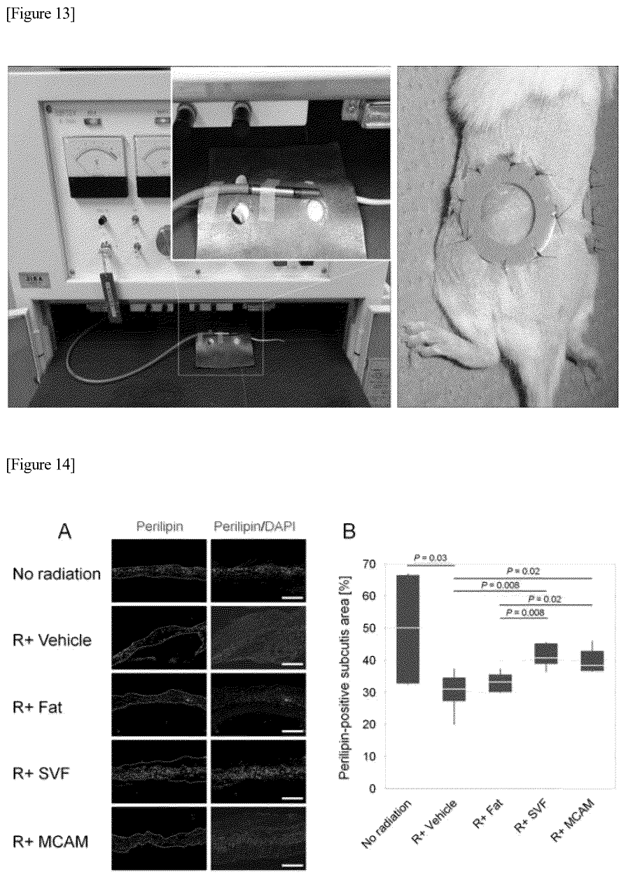

All animal procedures were performed in accordance with relevant guidelines and were approved by the Animal Experimentation Committee of Kyoto Prefectural University of Medicine. Eight-week-old male nude mice were selected for the study. Under general anesthesia (sevoflurane inhalation) and full body shielding (1 mm thick lead X-ray plate), a circular dorsal area (diameter 1.5 cm) of each mouse was irradiated using an M-150WE device (SOFTEX Japan Corp., Tokyo, Japan) (FIG. 13) (FIGS. 1-3). Four test groups (24 mice total) each received four weekly irradiations of 10 Gy (Table 1), while six mice (non-irradiated control group) received no radiation exposure (FIG. 13).

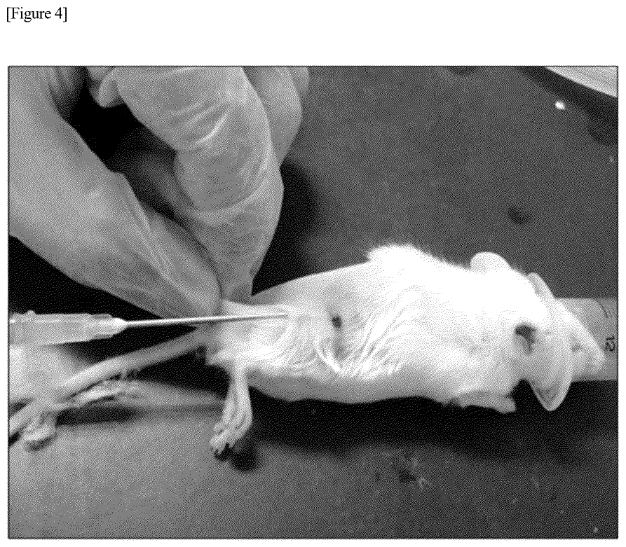

At the end of the fourth treatment, either medium alone or medium containing one of three adipose tissue-derived products was subcutaneously transplanted into the irradiated area as follows (Table 1 and FIG. 4).
0.2 mL of Dulbecco's Modified Eagle Medium (DMEM): Vehicle group;
0.2 mL DMEM containing 7.5×10⁴ SVF-derived cells: SVF group;
0.2 mL of centrifuged fat: Fat group; and
0.1 mL of fragmented MCAM in 0.1 mL DMEM: MCAM group

The specified materials for each group were injected subcutaneously into the mouse back using an 18-gauge needle attached to a 1 mL Luer-Lock syringe, approached via a subcutaneous tunnel starting 1 cm caudal to the point of injection, minimizing graft leakage (n=6 mice per group).

After 6 months, a 6 mm full-thickness punch biopsy was obtained for histological examination. A donut-shaped silicone splint (9 mm diameter) was set to prevent wound contracture and secured with instant adhesive and 6-0 nylon sutures under general anesthesia (sevoflurane inhalation) (FIG. 13). The wound was covered with anon-adherent dressing, wrapped in a transparent sterile bandage, and photographed on days 0, 3, 6, 9, 12, and 15. The wound surface area was calculated using ImageJ software (Bethesda, Maryland, USA).

TABLE 1

| | | Radiation | |
|---|---|---|---|
| Group | | Dose (Gy)* | Injection Material |
| Non-irradiated group | | 0 | DMEM (0.2 mL) |
| Irradiated | Vehicle group | 40 (10 × 4) | DMEM (0.2 mL) |
| group | Fat group | 40 (10 × 4) | Centrifuged adipose tissue (0.2 mL) |
| | SVF group | 40 (10 × 4) | Adipose tissue-derived cells from SVF in |
| | | | DMEM (7.^{5×104}cells in 0.2 mL) |
| | MCAM group | 40 (10 × 4) | Fragmented adipose tissue matrix |
| | | | (7.5 × 10⁴ cells in 0.2 mL) |

0.2 mL of MCAM yields approximately 5 to 10×10⁴ cells through collagenase enzymatic treatment; therefore, 0.2 mL of 7.5×10⁴ SVF cells was used in the SVF group.
DMEM, Dulbecco's Modified Eagle Medium;
SVF, stromal vascular fraction;
MCAM, minced adipose tissue matrix.
*Total dose (fraction dose×number of fractions).
**0.2 mL aliquot administered.
All fat components are human-derived.

### <Histological Examination and Immunohistochemical Staining>

Irradiated skin samples were immersed in zinc formalin fixative (Sigma-Aldrich) and embedded in paraffin. Sections (5 μm) were stained with hematoxylin and eosin, Masson's trichrome, and immunohistochemical staining as described below Tissue sections were blocked for 1 hour in 0.3% Triton X-100/PBS containing 5% goat serum (Nacalai Tesque, Kyoto, Japan) in 0.3% Triton X-100/PBS for 1 hour, then incubated with rabbit anti-human perilipin antibody (1:500) (Cell Signaling Technology, Danvers, MA), followed by incubation with a secondary antibody conjugated to Alexa Fluor 488 (Thermo Fisher Scientific, Waltham, MA). Cell nuclei were stained with DAPI (Thermo Fisher Scientific). All samples were observed and analyzed using a BIOREVO microscope (BZ-9000; Keyence, Osaka, Japan), MZ-II Analyzer software (Keyence), and an LSM510 confocal microscope (Carl Zeiss, Ober-Kochen, Germany).

### <Statistical Analysis>

Results were subjected to nonparametric analysis. Differences between groups were analyzed using the Kruskal-Wallis and Mann-Whitney tests in the JMP statistical program (SAS Institute, Cary, NC). Data are presented as median and interquartile range (IQR). A p-value less than 0.05 was considered statistically significant.

### (2) Results

<Preventive Effect of Adipose Tissue-Derived Products on Wound Healing Capacity after Radiation Therapy>

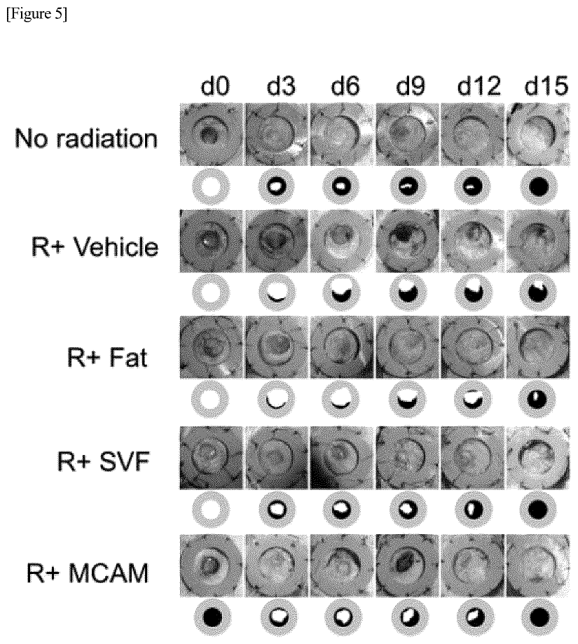

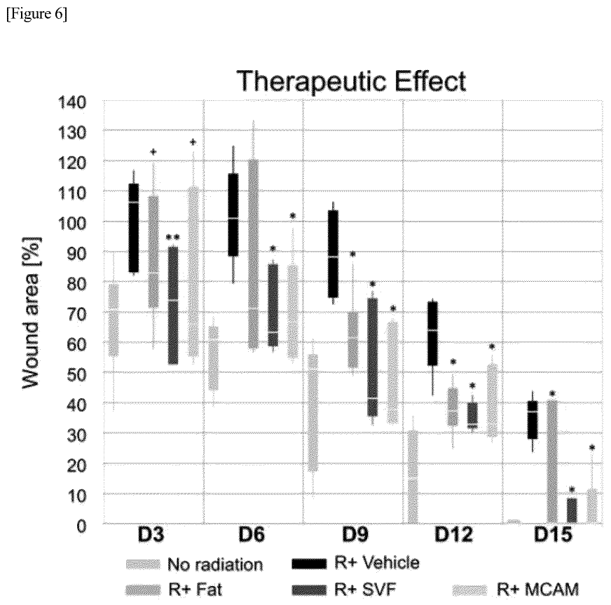

To evaluate the therapeutic effect on wound healing capacity after radiation therapy, the prophylactic application of human adipose tissue-derived products were tested. The materials used were (1) centrifuged adipose tissue (fat), (2) SVF, and (3) MCAM (Table 1). In the control radiation group (DMEM vehicle treatment), wounds showed no change during the first 5 days, exhibited significantly delayed healing compared to the non-irradiated control, and demonstrated chronic radiation injury after a total radiation dose of 40 Gy (FIG. 5). Compared to the vehicle-treated radiation group, the prophylactic application of the three different materials significantly accelerated wound healing to levels equivalent to the non-irradiated control by day 15 (day 15, Fat, P=0.04; SVF, P=0.01; MCAM, P=0.02) to levels comparable to the non-irradiated control by day 15. These results indicate that adipose tissue-derived products containing ASCs protect against chronic radiation injury when applied after radiation therapy (FIG. 6). No significant differences were observed between the three types of adipose-derived products examined here in terms of their preventive effects.

<Histological Evaluation of Skin Samples 6 Months after Radiation Therapy>

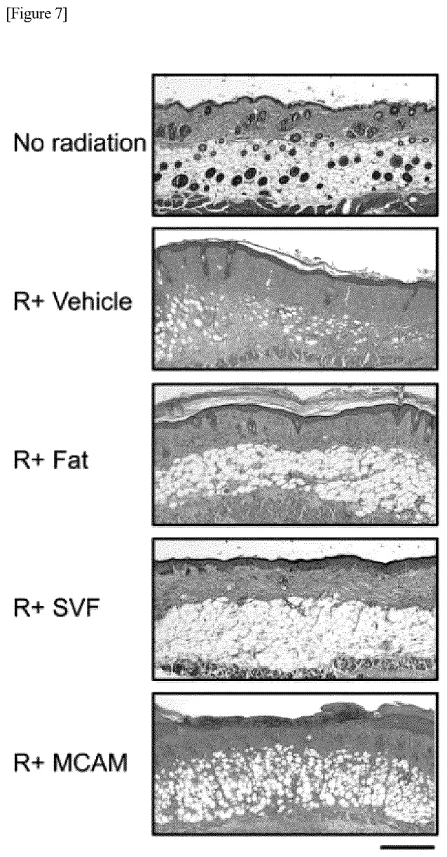

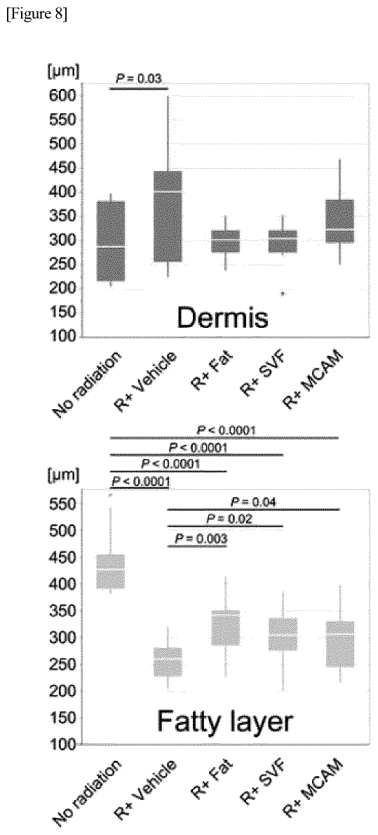

Irradiated skin injected with adipose tissue-derived products was histologically analyzed 6 months post-irradiation. In the control radiation group (solvent), keratinization of the epidermis and thickening of the spinous layer were observed in some areas (FIG. 7). However, injection of adipose tissue-derived product maintained skin structure similar to the non-irradiated control group. Furthermore, compared to the non-irradiated group, the solvent-treated group showed significant dermal hypertrophy (P=0.02). Since this effect is atypical skin change following radiation exposure, it was examined in the groups administered the entire adipose tissue-derived product. It was found to be reduced in all cases (Fat, P=0.7; SVF, P=0.7; MCAM, P=0.1; compared to the non-irradiated group) (FIG. 8).

The solvent-treated radiation group showed substantial atrophy of the fat layer compared to the non-irradiated control group. However, radiation-induced atrophy of the fat layer was significantly reduced in all groups treated with adipose tissue-derived product (Fat, P=0.003; SVF, P=0.02; MCAM, P=0.04), but these effects did not reach the level of the non-irradiated control group (FIG. 8). In the fat-treated groups, coarse adipose tissue with large fat droplets along the fascia was observed compared to the SVF-treated and MCAM-treated groups (FIG. 7).

Immunohistochemical evaluation to determine the presence of surviving adipocytes (FIG. 14) showed that the areas filled with surviving adipocytes (peripin-positive cells) in the subcutaneous layer of the fat-treated group were smaller than those in the SVF-treated and MCAM-treated groups.

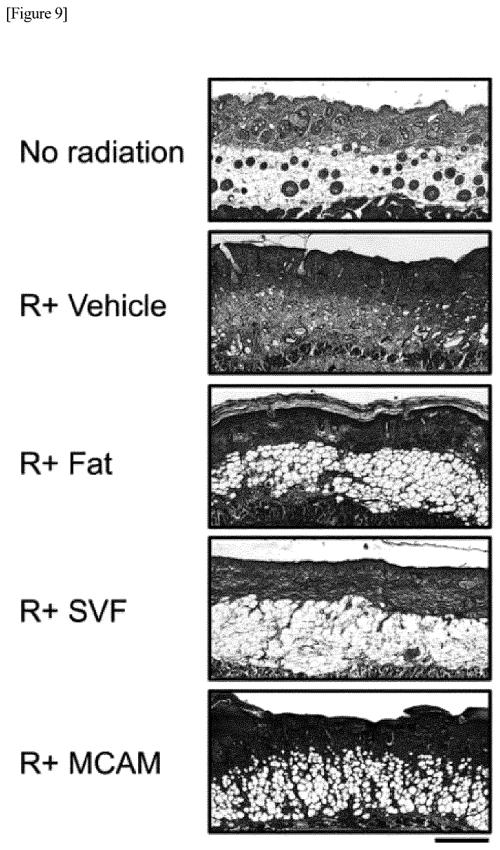

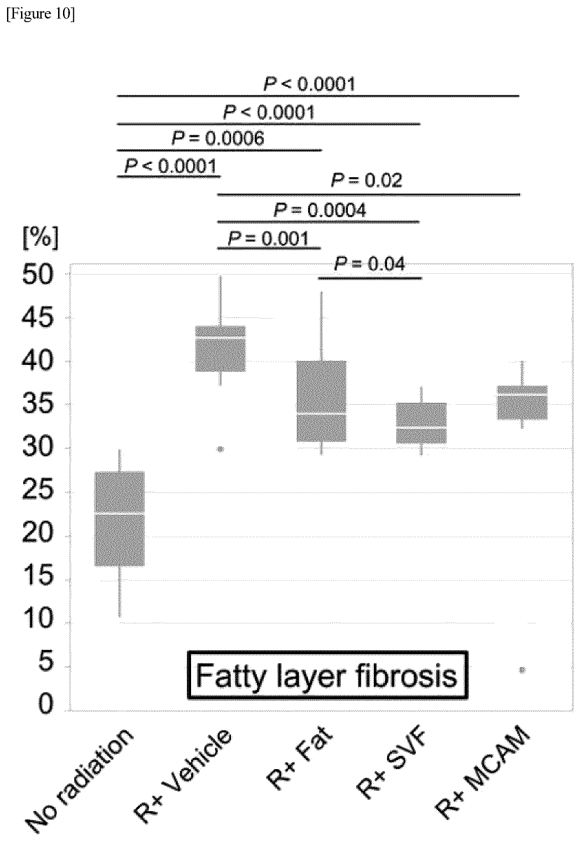

Histological observation using Masson's trichrome staining revealed not only adipose tissue atrophy but also the presence of fibrous deposits in the fat layer in the total irradiation group compared to the non-irradiated control group. However, the degree of fibrosis in the adipose layer did not reach the level of the non-irradiated control group (FIG. 10), and was significantly less in all adipose tissue-derived preparation-treated groups compared to the solvent-treated group (Fat, P=0.01; SVF, P=0.0004; MCAM, P=0.02) (FIG. 9).

These results indicate that subcutaneous atrophy was significantly prevented in all adipose tissue-derived product treatment groups compared to the vehicle treatment group, with this effect being more pronounced in the SVF and MCAM treatment groups than in the fat treatment group.

### (3) Summary

Radiation therapy often induces short-term erythema and tissue damage that can progress to fibrosis, atrophy, and ischemia over the long term (Non-Patent Documents 17-19). Fractionated irradiation protocols are now commonly used to improve the efficacy of cancer treatment and reduce associated complications, but long-term radiation damage still frequently occurs. One serious problem associated with delayed radiation injury is that irradiated skin loses its wound-healing capacity, necessitating curative treatment during the late phase (Non-Patent Documents 20-23). Once skin ulcers develop, they are refractory and require extensive use of resources. No treatment exists for patients affected by this type of late radiation injury. Therefore, a preventive treatment capable of maintaining the healing capacity of irradiated tissue is strongly desired.

ASC-based cell transplantation therapy has been reported as a promising regenerative treatment with multiple advantages, including ease of isolation, abundance, and anti-inflammatory, anti-apoptotic, and pro-angiogenic effects (Non-Patent Document 10). Previous studies have shown that injecting liposuctioned material or adipose tissue-derived products containing ASC can activate the pathological state of tissues and accelerate wound healing (Non-Patent Document 9, 14, 24-27).

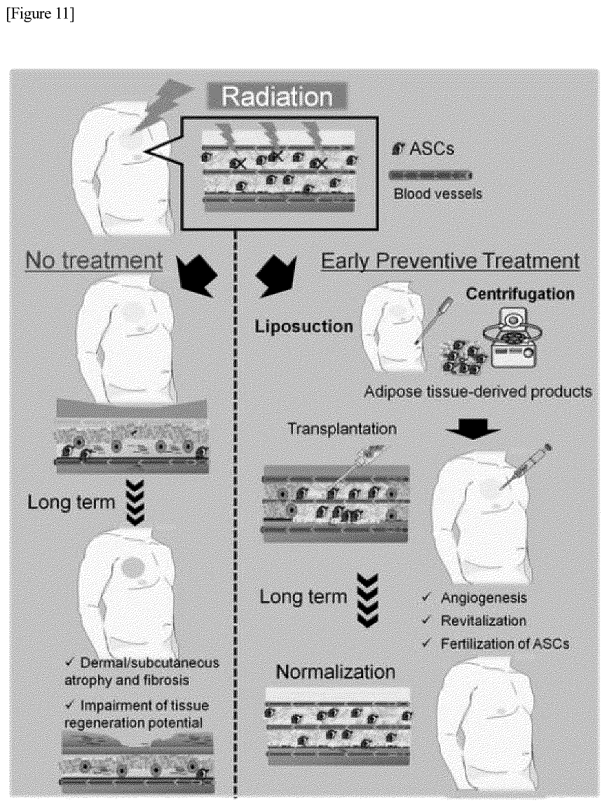

In the present invention, we tested whether ASCs and two types of adipose tissue-derived products have therapeutic effects in preventing radiation-induced tissue damage. In the experimental model used in the examples of the present invention, severe symptoms of acute tissue damage, such as ulcer formation, were avoided. At six months, severe atrophy and fibrosis of the skin and subcutaneous tissue, along with delayed wound healing, were observed, appropriately reproducing the common course following radiation therapy. Using this radiation therapy model, it was demonstrated that administering stem cells or stem cell-related substances immediately after radiation exposure can prevent radiation damage. Specifically, administering fat, SVF, or MCAM early after radiation therapy was shown to improve wound healing at six months, and administering adipose tissue-derived substances containing ASCs was shown to prevent chronic radiation damage (FIG. 11).

1-7. (canceled)

8. A method for protecting against adiation damage, which comprises administering adipose tissue or adipose tissue-derived product to a subject.

9. The method of claim 1, wherein the radiation damage is chronic radiation damage.

10. The method of claim 1, wherein the radiation damage is a decrease in wound healing ability.

11. The method of claim 1, wherein the adipose tissue or adipose tissue-derived product has an action to prevent subcutaneous tissue atrophy.

12. The method of claim 1, wherein the fat tissue-derived product is stromal vascular fraction (SVF), micronized cellular adipose matrix (MCAM), or adipose-derived stem/stromal cells (ASC), or a culture supernatant of SVF, MCAM or ASC, or an extract of SVF, MCAM or ASC.

13. The method of claim 1, wherein the adipose tissue or adipose tissue-derived product is administered to a subject immediately after radiation exposure.

14. The method of claim 1, wherein the adipose tissue or the adipose tissue-derived product is autologous or allogeneic.
